(19) United States
(12) Patent Application Publication
(10) Pub. No.: US 2014/0221780 A1
(43) Pub. Date: Aug. 7, 2014

(54) COMPLEXITY BASED METHODS AND SYSTEMS FOR DETECTING DEPRESSION

(71) Applicants: Ary L. Goldberger, Newton Center, MA (US); Madalena Damasio Costa, Brookline, MA (US)

(72) Inventors: Ary L. Goldberger, Newton Center, MA (US); Madalena Damasio Costa, Brookline, MA (US)

(73) Assignee: PRESIDENT AND FELLOWS OF HARVARD COLLEGE, Cambridge, MA (US)

(21) Appl. No.: 14/233,999

(22) PCT Filed: Jul. 23, 2012
(86) PCT No.: PCT/US2012/047862

§ 371 Date: Apr. 21, 2014

(51) Int. Cl. A61B 5/16; A61B 5/0476; A61B 5/00; A61B 5/0205

(52) U.S. Cl. CPC A61B 5/165; A61B 5/0205; A61B 5/0476; A61B 5/4803
USPC 600/301; 600/300; 600/508; 600/544; 600/586

(57) ABSTRACT

Major depression can affect multiple physiologic systems. Analysis of signals that reflect integrated function may be useful in probing dynamical changes in this syndrome. Complex variability can be used as a marker of healthy, adaptive control mechanisms and dynamical complexity decreases with aging and disease. The heart rate (HR) dynamics in non-medicated, young to middle-aged males during an acute major depressive episode exhibit lower complexity compared with healthy counterparts. By analyzing HR time series, a neuroautonomically regulated signal, during sleep, using the multiscale entropy method, a measure of complexity of HR dynamics can be determined. The complexity of the HR dynamics is significantly lower for depressed than for non-depressed subjects for the entire night and combined sleep stages 1 and 2, providing an indication of depression. These complexity signals, individually, or in combination with the complexity of other physiologic signals, can be used to define novel dynamical biomarkers of depression.

### CROSS-REFERENCE TO RELATED APPLICATIONS

This application claims any and all benefits as provided by law of U.S. Provisional Application No. 61/510,937 filed Jul. 22, 2011, entitled “Complexity Based Methods and Systems for Detecting Depression,” which is hereby incorporated by reference in its entirety.

This application is related to U.S. Pat. Nos. 7,601,124 and 7,882,167, the entire contents of which are hereby incorporated by reference.

### STATEMENT REGARDING FEDERALLY SPONSORED RESEARCH

This invention was made with government support under grant no. K99 AG030677 awarded by the National Institute on Aging and no. U01 EB008577 awarded by the National Institute of Health. The government has certain rights in the invention.

### REFERENCE TO MICROFICHE APPENDIX

Not applicable

### BACKGROUND

1. Technical Field of the Invention

The present invention is directed to methods and systems for diagnosing depression and similar illness using complexity analysis of physiologic signals. More specifically, complexity analysis of physiologic signals, such as, heart rate, voice/speech, and brain waves, to determine biomarkers for neurologic disease, such as depression.

2. Description of the Prior Art

Major depression (MD) is highly prevalent and a leading cause of disability worldwide.¹⁻³ MD is a multisystem illness, affecting, for example, endocrine, immunological, nocioceptive and cardiovascular function.^{4,5} Patients have a twofold to fourfold increased risk of developing cardiovascular disease⁶ and of mortality after a cardiac event.⁷ One underlying mechanism could be related to dysregulation of the autonomic nervous system.^{8,9} However, the diagnosis of MD still lacks objective assays or genomic markers.¹⁰⁻¹²

Complexity-based dynamical analysis of heart rate has been used in detecting aging, pathology, toxicity and/or the efficacy of therapeutic intervention and treatment, typically for physical diseases, such as congestive heart failure and atrial fibrillation. This methodology has not been used in the prior art to detect and treat mental and neurologic disease.

### SUMMARY

The present invention is directed to method and systems for quantifying complexity of physiologic signals for use as an indicator or biomarker for mental and neurologic disease, such as Major Depression (MD). In accordance with the invention, the complexity of physiologic signals including heart rate, heart rate variation, brain waves and voice can be used to indicate mental and neurologic disease and as an indicator of the effectiveness of treatment.

In accordance with various embodiments of the invention, data representing one or more physiologic signals can be produced and stored. The data can be processed to remove noise, outliers and other errors. The data from a given patient can be analyzed to quantify the dynamical complexity of the data and the dynamical complexity of the data can be compared to with a baseline representing the complexity of the dynamics of: i) a healthy subject, ii) the patient prior to a given therapeutic intervention, iii) the patient at any given past time after treatment onset. As indicated, the lower dynamical complexity can be used to indicate the presence of mental and neurologic disease. In some embodiments, more than one physiologic signal can be measured, and data representing those signals can be stored and analyzed. Where more than one signal is analyzed, each signal can be compared to a baseline representing the dynamical complexity for a healthy subject (or, in the case of a recovering subject, their prior signal data) and the data from each signal can be combined in a single dynamical complexity indicator that can be determined as a function of the data for each signal. Alternatively, each signal can be used individually to confirm the diagnosis.

To the extent that mental and neurologic disease affects multiple regulatory mechanisms operating over a wide range of time scales, diagnostic and therapeutic approaches can be enhanced by (i) probing signals that reflect integrated physiology, such as heart rate variability (HRV)^{13,14} and (ii) using newer measures that quantify dynamical properties on multiple time scales.¹⁵⁻¹⁷

Beat-to-beat heart rate (HR) fluctuations can be particularly relevant to the understanding of the pathophysiology of MD as they encode information about underlying neuroautonomic control.^{13,14} Previous reports¹⁸⁻²³ on HR dynamics in MD generally suggest decreased parasympathetic or increased sympathetic effects. However, the findings are not fully consistent and discrepancies are difficult to resolve because of the heterogeneity of the populations studied, confounding medication effects and differences in analytical tools.

In accordance with the invention, a study was conducted using a population of non-medicated, young to middle-aged men during an acute major depressive episode (MDE) and a healthy control group. The complexity of cardiac interbeat interval time series,¹⁵⁻¹⁷ for the entire night and at different sleep stages, was quantified to gain further insight into the effects of MDE on HR dynamics.

Complexity relates to the information content (structural ‘richness’) of a signal²⁴ that emerges from the nonlinear interactions among regulatory components. Two signals can have identical variance but different complexity values that reflect differences in their dynamical properties.

High multiscale complexity has been proposed as a generic feature of healthy dynamics.²⁵ In contrast, disease and aging, marked by degraded functionality and adaptability, have been characterized by loss of complexity.^{15,17,26,27} In accordance with the present invention, the study confirmed the hypothesis that MDE is associated with degradation in the complexity of HR dynamics, reflecting neuroautonomic perturbations.

Complexity of cardiac interbeat interval fluctuations during sleeping hours can be assessed using the multiscale entropy (MSE) method described in detail elsewhere.^{15,17} This method has been widely used in the analysis of physiologic time series.²⁸⁻³⁰ MSE quantifies the complexity of a signal by computing an entropy measure called sample entropy^{31,32} (“SampEn”) on different time scales. Of note, conventional single scale-based entropy and mutual information algorithms are irregularity measures, which yield higher values for uncorrelated random signals than for intrinsically complex ones. In contrast, MSE analysis reveals that time-varying signals such as cardiac interbeat intervals under healthy conditions are more complex than highly irregular ones generated by pathologic processes such as atrial fibrillation.

In accordance with one embodiment of the invention, the analysis of nocturnal HR time series can be determined from a continuous electrocardiogram (ECG) that is routinely recorded with polysomnography (PSG). The advantage of using a nocturnal HR time series is that the level of physical activity among sleeping subjects is likely more comparable than during waking hours.

In addition to the complexity analysis, conventional HRV analyses comprising time and frequency (Fourier-based) domain measures can also be conducted.³³ The latter require the data to be stationary and linear. Despite the fact that physiologic data are almost invariably non-stationary and non-linear, traditional HRV measures are among the most frequently computed. This data may or may not be relevant for diagnosis.

In addition to heart rate and heart rate variability, other physiologic measures, such as voice signals can be used to develop a dynamical biomarker in accordance with the present invention. In some embodiments, speech (either free-form unscripted or scripted) can be recorded and compiled. The same or similar multiscale entropy analysis can be used to quantify the dynamical complexity of the voice signals. The dynamical complexity can be compared with a baseline dynamical complexity to determine whether mental or neurologic disease is indicated.

Further, in accordance with the invention, the determination of dynamical complexity can include a tolerance component, such as a noise rejection level, that can be selected according to one or more predefined parameters. In accordance with one embodiment of the invention, the tolerance is determined as a function of the sampling period of data points collected.

In one aspect, provided herein is a method for detecting a mental disease comprising: measuring at least one physiological signal from a patient; determining a signal complexity index as a function of at least one of the physiological signals; comparing the signal complexity index to at least one of a baseline complexity index of a healthy subject, a baseline complexity index of the patient prior to treatment or a baseline complexity index of the patient at a past time to determine if the signal complexity index varies from the baseline complexity index by a threshold amount.

In one embodiment of this aspect, determining the signal complexity index includes determining an entropy value over multiple time scales.

In another embodiment of this aspect, determining the signal complexity includes determining a tolerance parameter r and the tolerance parameter r is determined as a function of a sampling frequency of at least one of the physiological signals.

In another embodiment of this aspect, the physiological signals are selected from the group including heart rate signals, brain wave signals and voice signals.

In another embodiment of this aspect, the method further comprises: developing a treatment plan for the mental disease based on the variation between the signal complexity index and the baseline complexity index.

In another embodiment of this aspect, the method further comprises: modifying an existing treatment plan for the mental disease based on the variation between the signal complexity index and the baseline complexity index.

In another embodiment of this aspect, the signal complexity index is a short time scale signal complexity index, wherein the short time scale signal complexity index corresponds with an area under a multiscale entropy curve from scales 1 to 8 corresponding to frequencies between 0.12 and 0.5 Hz, and wherein the short time scale signal complexity index is compared to an average short time scale complexity index for the healthy subject.

In another embodiment of this aspect, the mental disease is indicated when the short time scale signal complexity index of the patient is statistically significantly lower than the average short time scale complexity index for the healthy subject.

In another embodiment of this aspect, the mental disease is indicated when the short time scale signal complexity index of the patient is about 1.8 lower than the average short time scale complexity index for the healthy subject.

In another embodiment of this aspect, a lower signal complexity index indicates a more severe mental disease as compared to a higher signal complexity index.

In another embodiment of this aspect, the mental disease is indicated by a statistically significantly lower value of at least one of a mean of a heartbeat interval, a mean of a standard deviation of normal sinus to normal sinus (NN) intervals in all non-overlapping 5-minute segments, a percentage of adjacent NN intervals whose difference is higher than 10 ms and a spectral power in very low frequency and low frequency ranges.

In another embodiment of this aspect, the mental disease is major depression.

In another aspect, provided herein is a computer implemented method for detecting a mental disease, comprising: on a device having one or more processors and a memory storing one or more programs for execution by the one or more processors, the one or more programs including instructions for: measuring at least one physiological signal from a patient; determining a signal complexity index as a function of at least one of the physiological signals; comparing the signal complexity index to at least one of a baseline complexity index of a healthy subject, a baseline complexity index of the patient prior to treatment or a baseline complexity index of the patient at a past time to determine if the signal complexity index varies from the baseline complexity index by a threshold amount.

In one embodiment of this aspect, determining the signal complexity index includes determining an entropy value over multiple time scales.

In another embodiment of this aspect, determining the signal complexity includes determining a tolerance parameter r and the tolerance parameter r is determined as a function of a sampling frequency of at least one of the physiological signals.

In another embodiment of this aspect, the physiological signals are selected from the group including heart rate signals, brain wave signals and voice signals.

In another embodiment of this aspect, the computer implemented method further comprises: developing a treatment plan for the mental disease based on the variation between the signal complexity index and the baseline complexity index.

In another embodiment of this aspect, the method further comprises: modifying an existing treatment plan for the mental disease based on the variation between the signal complexity index and the baseline complexity index.

In another embodiment of this aspect, the signal complexity index is a short time scale signal complexity index, wherein the short time scale signal complexity index corresponds with an area under a multiscale entropy curve from scales 1 to 8 corresponding to frequencies between 0.12 and 0.5 Hz, and wherein the short time scale signal complexity index is compared to an average short time scale complexity index for the healthy subject.

In another embodiment of this aspect, the mental disease is indicated when the short time scale signal complexity index of the patient is statistically significantly lower than the average short time scale complexity index for the healthy subject.

In another embodiment of this aspect, the mental disease is indicated when the short time scale signal complexity index of the patient is about 1.8 lower than the average short time scale complexity index for the healthy subject.

In another embodiment of this aspect, a lower signal complexity index indicates a more severe mental disease as compared to a higher signal complexity index.

In another embodiment of this aspect, the mental disease is indicated by a statistically significantly lower value of at least one of a mean of a heartbeat interval, a mean of a standard deviation of normal sinus to normal sinus (NN) intervals in all non-overlapping 5-minute segments, a percentage of adjacent NN intervals whose difference is higher than 10 ms and a spectral power in very low frequency and low frequency ranges.

In another embodiment of this aspect, the mental disease is major depression.

In another aspect, provided herein is a computer system for detecting a mental disease, comprising: one or more processors; and memory to store: one or more programs, the one or more programs comprising: instructions for: measuring at least one physiological signal from a patient; determining a signal complexity index as a function of at least one of the physiological signals; comparing the signal complexity index to at least one of a baseline complexity index of a healthy subject, a baseline complexity index of the patient prior to treatment or a baseline complexity index of the patient at a past time to determine if the signal complexity index varies from the baseline complexity index by a threshold amount.

In one embodiment of this aspect, determining the signal complexity index includes determining an entropy value over multiple time scales.

In another embodiment of this aspect, determining the signal complexity includes determining a tolerance parameter r and the tolerance parameter r is determined as a function of a sampling frequency of at least one of the physiological signals.

In another embodiment of this aspect, the physiological signals are selected from the group including heart rate signals, brain wave signals and voice signals.

In another embodiment of this aspect, the one or more programs further comprise instructions for: developing a treatment plan for the mental disease based on the variation between the signal complexity index and the baseline complexity index.

In another embodiment of this aspect, the method further comprises: modifying an existing treatment plan for the mental disease based on the variation between the signal complexity index and the baseline complexity index.

In another embodiment of this aspect, the signal complexity index is a short time scale signal complexity index, wherein the short time scale signal complexity index corresponds with an area under a multiscale entropy curve from scales 1 to 8 corresponding to frequencies between 0.12 and 0.5 Hz, and wherein the short time scale signal complexity index is compared to an average short time scale complexity index for the healthy subject.

In another embodiment of this aspect, the mental disease is indicated when the short time scale signal complexity index of the patient is statistically significantly lower than the average short time scale complexity index for the healthy subject.

In another embodiment of this aspect, the mental disease is indicated when the short time scale signal complexity index of the patient is about 1.8 lower than the average short time scale complexity index for the healthy subject.

In another embodiment of this aspect, a lower signal complexity index indicates a more severe mental disease as compared to a higher signal complexity index.

In another embodiment of this aspect, the mental disease is indicated by a statistically significantly lower value of at least one of a mean of a heartbeat interval, a mean of a standard deviation of normal sinus to normal sinus (NN) intervals in all non-overlapping 5-minute segments, a percentage of adjacent NN intervals whose difference is higher than 10 ms and a spectral power in very low frequency and low frequency ranges.

In another embodiment of this aspect, the mental disease is major depression.

In another aspect, provided herein is a non-transitory computer-readable storage medium storing one or more programs configured to be executed by one or more processing units at a computer comprising: instructions for: measuring at least one physiological signal from a patient; determining a signal complexity index as a function of at least one of the physiological signals; comparing the signal complexity index to at least one of a baseline complexity index of a healthy subject, a baseline complexity index of the patient prior to treatment or a baseline complexity index of the patient at a past time to determine if the signal complexity index varies from the baseline complexity index by a threshold amount.

In one embodiment of this aspect, determining the signal complexity index includes determining an entropy value over multiple time scales.

In another embodiment of this aspect, determining the signal complexity includes determining a tolerance parameter r and the tolerance parameter r is determined as a function of a sampling frequency of at least one of the physiological signals.

In another embodiment of this aspect, the physiological signals are selected from the group including heart rate signals, brain wave signals and voice signals.

In another embodiment of this aspect, the one or more programs further comprise instructions for: developing a treatment plan for the mental disease based on the variation between the signal complexity index and the baseline complexity index.

In another embodiment of this aspect, the method further comprises: modifying an existing treatment plan for the mental disease based on the variation between the signal complexity index and the baseline complexity index.

In another embodiment of this aspect, the signal complexity index is a short time scale signal complexity index, wherein the short time scale signal complexity index corresponds with an area under a multiscale entropy curve from scales 1 to 8 corresponding to frequencies between 0.12 and 0.5 Hz, and wherein the short time scale signal complexity index is compared to an average short time scale complexity index for the healthy subject.

In another embodiment of this aspect, the mental disease is indicated when the short time scale signal complexity index of the patient is statistically significantly lower than the average short time scale complexity index for the healthy subject.

In another embodiment of this aspect, the mental disease is indicated when the short time scale signal complexity index of the patient is about 1.8 lower than the average short time scale complexity index for the healthy subject.

In another embodiment of this aspect, a lower signal complexity index indicates a more severe mental disease as compared to a higher signal complexity index.

In another embodiment of this aspect, the mental disease is indicated by a statistically significantly lower value of at least one of a mean of a heartbeat interval, a mean of a standard deviation of normal sinus to normal sinus (NN) intervals in all non-overlapping 5-minute segments, a percentage of adjacent NN intervals whose difference is higher than 10 ms and a spectral power in very low frequency and low frequency ranges.

In another embodiment of this aspect, the mental disease is major depression.

These and other capabilities of the invention, along with the invention itself, will be more fully understood after a review of the following figures, detailed description, and claims.

### BRIEF DESCRIPTION OF THE FIGURES

The accompanying drawings, which are incorporated into this specification, illustrate one or more exemplary embodiments of the inventions disclosed herein and, together with the detailed description, serve to explain the principles and exemplary implementations of these inventions. One of skill in the art will understand that the drawings are illustrative only, and that what is depicted therein may be adapted based on the text of the specification and the spirit and scope of the teachings herein.

In the drawings, where like reference numerals refer to like reference in the specification:

FIG. 1 shows a graph of a Multiscale Entropy Analysis of HRV for 1 to 20 time scales according to an embodiment of the invention.

FIG. 2 shows a diagram of a voice and local energy time series for both depressive and normal (remission) phases according to an embodiment of the invention.

FIG. 3 shows a graph of a Multiscale Entropy Analysis of voice local energy for 1 to 20 time scales according to an embodiment of the invention.

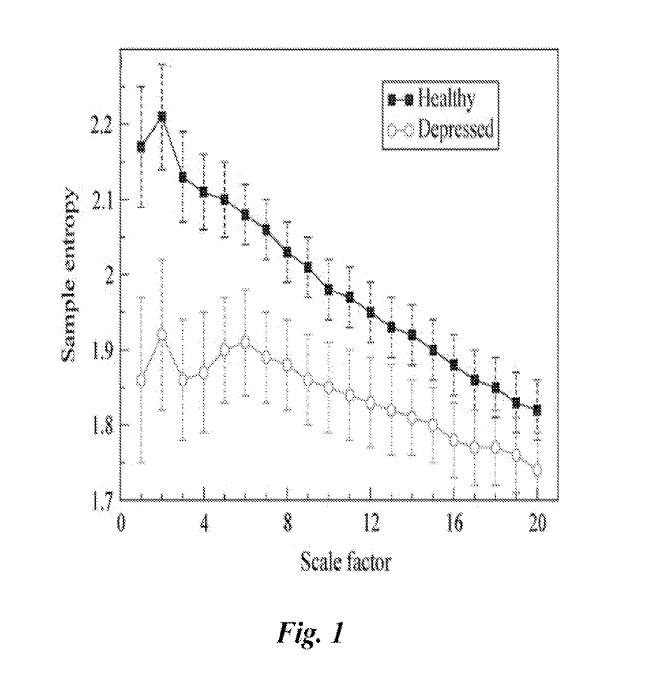

Table 1 shows select demographic and clinical data for the sample used to generate the Multiscale Entropy Analysis graph of FIG. 1.

Table 2 shows select electroencephalography (EEG) sleep data summaries for the sample used to generate the Multiscale Entropy Analysis graph of FIG. 1.

Table 3 shows select heart rate variability analysis data for the sample used to generate the Multiscale Entropy Analysis graph of FIG. 1.

### DETAILED DESCRIPTION OF PREFERRED EMBODIMENTS

It should be understood that this invention is not limited to the particular methodology, protocols, etc., described herein and as such may vary. The terminology used herein is for the purpose of describing particular embodiments only, and is not intended to limit the scope of the present invention, which is defined solely by the claims.

As used herein and in the claims, the singular forms include the plural reference and vice versa unless the context clearly indicates otherwise. Other than in the operating examples, or where otherwise indicated, all numbers expressing quantities used herein should be understood as modified in all instances by the term “about.”

All publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

Some Selected Definitions

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments of the aspects described herein, and are not intended to limit the claimed invention, because the scope of the invention is limited only by the claims. Further, unless otherwise required by context, singular terms shall include pluralities and plural terms shall include the singular.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Other than in the operating examples, or where otherwise indicated, all numbers expressing quantities used herein should be understood as modified in all instances by the term “about.” The term “about” when used in connection with percentages may mean±1%.

The singular terms “a,” “an,” and “the” include plural referents unless context clearly indicates otherwise. Similarly, the word “or” is intended to include “and” unless the context clearly indicates otherwise. Thus for example, references to “the method” includes one or more methods, and/or steps of the type described herein and/or which will become apparent to those persons skilled in the art upon reading this disclosure and so forth.

Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments of the aspects described herein, the subject is a mammal, e.g., a primate, e.g., a human. The terms, “patient” and “subject” are used interchangeably herein.

In some embodiments, the subject is a mammal. The mammal can be a human, non-human primate, mouse, rat, dog, cat, horse, or cow, but are not limited to these examples. Mammals other than humans can be advantageously used as subjects that represent animal models of disorders.

A subject can be one who has been previously diagnosed with or identified as suffering from or having a disease or disorder caused by any microbes or pathogens described herein. By way of example only, a subject can be diagnosed with sepsis, inflammatory diseases, or infections.

To the extent not already indicated, it will be understood by those of ordinary skill in the art that any one of the various embodiments herein described and illustrated may be further modified to incorporate features shown in any of the other embodiments disclosed herein.

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

The present invention is directed to method and systems for quantifying complexity of physiologic signals for use as an indicator or biomarker for mental and neurologic disease, such as Major Depression (MD). In accordance with the invention, the complexity of physiologic signals including heart rate (e.g., ECG), brain waves (e.g., EEG) and voice can be used to indicate mental and neurologic disease and as an indicator of the effectiveness of treatment.

### Subjects and Methods

In accordance with one embodiment of the invention, a total of 20 control subjects and 25 unmedicated male inpatients with an acute episode of MD, according to Diagnostic and Statistical Manual of Mental Disorders, Fourth Edition, text revision (DSM-IV-TR) criteria, were evaluated. Table 1 shows selected demographic and clinical data for this evaluation. Healthy controls were recruited from the community; patients were recruited from both the Sleep Laboratory and an inpatient psychiatry ward at Erasme Academic Hospital in Brussels. Controls were determined to be free of DSM-IV-TR axis I or axis II diagnoses and had no family history of major psychiatric disorders. Subjects reported a regular sleep-wake schedule and no current or past sleep disorders. Depressive symptom severity was assessed with the 24-item Hamilton rating scale for depression.³⁴

Patients were included if they had the following: (1) a Hamilton rating scale for depression score of 20 or greater;³⁴ (2) were not taking psychotropic medications and (3) had a Pittsburgh Sleep Quality Index of five or greater.³⁵ Subjects were excluded if they had untreated or poorly controlled conditions known to confound analysis of the sleep electroencephalogram and/or HRV results, for example, diabetes mellitus and chronic heart failure.

In addition, subjects who required treatment with agents known to affect either signal (for example, β-blockers or corticosteroids) were also excluded. Both controls and patients were medically screened with complete physical examination, chest X-ray, 12-lead ECG, EEG and standard laboratory tests as well as blood and urine toxicology screens. They did not show evident cardiovascular or endocrine abnormalities, primary sleep disorders and had a body mass index <29 kg m². Before signing informed consent, each subject received a detailed description of the procedures involved in the study and was deemed capable. The protocol was approved by the Ethics Committee of Erasme Academic Hospital, Free University of Brussels. A more complete description of our recruitment procedures is provided elsewhere.³⁶

### Recordings and Experimental Conditions.

In the patient group, sleep studies were conducted after at least a 3-week, psychotropic medication-free evaluation period. PSG recordings were obtained during two to three consecutive nights, of which, only the last one was examined in this study.³⁷ HR parameters were obtained from analysis of cardiac interbeat intervals from the continuous ECG recorded as part of the PSG exams, (Alice 5 Diagnostic Sleep System, Philips Respironics, Murrysville, Pa., USA). Nineteen lead electroencephalograms were recorded according to the International 10-20 Standard, with a contralateral reference to the A1 or A2 mastoid derivation, along with two electrooculograms and one submental electromyogram, as previously described.³⁶ Using a customized program (Endymion 1993-2009, Sleep Laboratory, Erasme Hospital) to facilitate analysis, each 20-second PSG epoch was visually scored according to standard criteria.³⁸

### HRV Computations.

From the European data format (edf) files, the ECG signal was extracted and converted to open source WaveForm DataBase (WFDB) format (http://www.physionet.org). An automated Q-wave, R-wave and S-wave (QRS) detection algorithm was then used to detect beats and annotate them as either normal sinus or ectopic.³⁹ Outliers due to missed or false beat detections were identified using a sliding window average filter. Intervals <0.4 s or greater than 2.0 s were excluded from the window average. Next, using a window of 41 intervals, the average over the window was calculated, excluding the central interval. If the central interval was outside 20% of the window average this interval was excluded and the window advanced by one interval.

From the resulting beat annotation files, the following standard time domain HRV statistics were calculated: the average of all the normal sinus to normal sinus (NN) intervals (AVNN), the standard deviation (s.d.) of all NN intervals (SDNN), the s.d. of the averages of NN intervals in all 5-min segments (SDANN), the mean of the s.d.'s of NN intervals in all 5-min segments (SDNNINDX), the root mean square of consecutive differences between adjacent NN intervals (rMSSD), and the percentage of adjacent intervals whose difference is higher than 10 ms (pNN10), 25 ms (pNN25) and 50 ms (pNN50)⁴° (http://www.physionet.org).

The following standard frequency domain measures were calculated using the Lomb periodogram for unevenly sampled data: total spectral power (TOTPWR; 0-0.4 Hz), ultra-low frequency power (ULF; 0-0.003 Hz), very-low frequency power (VLF; 0.003-0.04 Hz), low frequency power (LF; 0.04-0.15 Hz), high frequency power (HF; 0.15-0.4 Hz), the ratio of low to high frequency power (LF/HF) and the slope (b) of the spectrogram on a log-log scale assessed over the range of 0 to 0.04 Hz.

SDNN and TOTPWR are measures of variance. SDANN is a measure of the degree of non-stationarity of the time series. SDNNINDX quantifies how much the variance changes over time. rMSSD and pNN measures quantify HF fluctuations. Therefore, these time domain measures, in addition to HF power, have been used as indexes of cardiac vagal tone modulation. LF power is thought to reflect both sympathetic and vagal influences. LF/HF ratio has been proposed as an index of cardiac autonomic control but it is no longer widely accepted as a direct indicator of ‘sympatho-vagal balance.’ ULF and VLF powers quantify nonspecific trends in the time series. The b exponent is one measure of the fractal scaling properties of a signal. For HR time series obtained from healthy subjects under baseline conditions, this exponent is usually close to 1, which indicates the presence of long-range correlations.^{41,42}

### Complexity Analysis.

In accordance with one embodiment of the invention the dynamical complexity of the cardiac interbeat (RR) interval time series can be determined using the MSE method described in detail in U.S. Pat. Nos. 7,601,124 and 7,882,167 as well as in references 15 and 17 identified below, all of which are hereby incorporated by reference in their entirety. The MSE method quantifies the degree of irregularity of a signal using an entropy measure, such as SampEn, over multiple time scales. SampEn³¹ is the negative natural logarithm of an estimate of the conditional probability that subseries (epochs) of length m that match point-wise within a tolerance r will also match when the length of each of these subseries increases from m to m −1 data points. Signals that are highly irregular, and therefore more entropic, over a wide range of scales are more complex than both those that are highly regular, that is, periodic, and those that are irregular only at a single time scale (for example, white noise). The line obtained by connecting the SampEn values (y axis) for a range of time scales (x axis) is called the MSE curve. We then derived a short-term and longer-term complexity index (CI). In alternative embodiments, the sample entropy can be determined using well known Approximate Entropy equations, as discussed in reference 31.

As noted, in traditional HRV analysis, the cutoff separating the low and high frequency bands is 0.15 Hz, which corresponds to a period of ˜7 s. Physiologically, the high frequency band encompasses HR fluctuations associated with respiration. To quantify the complexity of the dynamics over a comparable high-frequency band, a short-term CI can be defined as the area under the MSE curve ranging from scales 1 to 8, inclusive (Given the fact that the average HR for both groups was about 60 beats per minute, the mean RR interval was ˜1 s and, therefore, scale n corresponds to ˜n seconds). It is noted that the specific cutoffs for both traditional frequency and complexity analyses are somewhat arbitrary.

To probe the complexity of the dynamics on relatively longer time scales, a long term CI can be defined as the area under the MSE curve from scales 1 to 20. The upper cutoff scale chosen (20), although necessarily arbitrary, is based on previous studies of HR time series.^{17,30} In the prior art, parameter values for calculating SampEn are typically selected as follows: m=2 and r=15% of the time series's.d.'s.^{31,32} In accordance with some embodiments of the invention, the parameter values can be chosen as follows: m=2 and r=8 ms. In accordance with the invention, the parameter r determines the level of noise accepted. For example, if |xᵢ−xᵢ₊₁|>r, then the two data points, x, and xᵢ₋₁, are distinguishable. Alternatively, if instead, |xᵢ−xᵢ₋₁|≦r, the two data points are indistinguishable, that is, their difference is considered noise, not signal. As the ECG recordings were sampled at 250 Hz, each RR interval is determined with an uncertainty of 4 ms ( 1/250), and the difference between RR intervals with an uncertainty of twice this value. To be above the noise level a value of r=8 ms was selected. Qualitatively similar results were obtained using r=15% of the time series's.d.'s, corresponding to r values ranging from 6 to 18 ms.

In this embodiment, the parameter r can be determined as function of the sampling period of HR time series. The parameter r can be any value larger than the inverse of signal's sampling frequency (i.e. the sampling period). In some embodiments, the parameter r can be equal to 1, 1.5, 2, 2.5, 3, 4 or 5 or more, times the sampling period, for example, of the HR time series. Preferably, the parameter r is approximately 2 or 3 times the sampling period. In alternative embodiments, other sources of noise can be identified and the parameter r can be determined as a function of these sources of noise. In some embodiments of the invention, the parameter r can be determined as a function of combination of factors. In other embodiments, multiple time series can be analyzed using different parameter r values, each determined as a function of a different source of noise.

The number of time scales one can probe depends on the total length of the original signal. Analysis of larger time scales requires longer signals. As a rule of thumb,³² at least 200 consecutive data points are needed for reliable calculation of SampEn. Therefore, if N is the length of a signal and S the largest time scale to be included in the MSE analysis, the relationship between the two variables should be such that N/S>200. For the analysis of HR dynamics during different sleep stages, segments of 15 min or longer with at least 1000 data points can be considered, and computed SampEn for time scales 1 to 5, inclusive. The analysis of full-night HR dynamics is not constrained by the length of the time series, which ranged from about 23 000 to 40 000 data points. Therefore, in accordance with the invention, a CI encompassing entropy values for both short (1 to 8) and longer (1 to 20) time scales can be determined.

In accordance with one embodiment of the invention, a computerized system can be used to process the HR data and determine various CI values. The computerized system can include a general purpose computer system having one or more processing units (e.g. an Intel, AMD or ARM cpu) and associated memories (e.g., volatile and non-volatile memory devices). The HR data can be stored in a file or a database and accessed by one or more computer programs to calculate the SampEn and other complexity and entropy parameters used to determine signal complexity. The input parameters, such as the tolerance r and length m can stored in an input file or input through a computer user interface. In some embodiments, the computer can store default values for the input parameters during setup and those values can be used until changed. Alternatively, the input parameters can be determined from other data or parameters, such as the sampling period of the HR data or by determining the average or standard deviation of some or all of the input data.

### Biostatistical Analyses.

Mann-Whitney non-parametric U-tests can be used to examine group (controls versus depression) differences in clinical, demographic, and sleep measures as well. This test can be deemed appropriate according to the sample size and data distribution, but other tests can be used depending on the sample size and distribution. To adjust for age, a least squares regression model can be used. To evaluate the association between MSE and depression during specific sleep stages, linear regression models can be used. In situations there were multiple observations per participant, generalized estimating equations methods with an exchangeable correlation structure can be used to account for within-participant correlation. Spearman correlation can be used to assess the association between MSE and the Hamilton depression scale among the depressed patients. All analyses can be performed with a (type I error) set at 0.05 using the SAS statistical software (version 9.13 for Windows, SAS Institute, Cary, N.C., USA).

### Results

Selected demographic and clinical data are summarized in Table 1.

### PSG Analyses.

Standard PSG measures are summarized in Table 2. Consistent with previous reports,^{43,44} the depression versus healthy groups showed statistically significant decreases in sleep efficiency, total sleep time, rapid eye movement latency, percentage delta sleep, and increases in sleep onset latency, percentage stage 1 sleep, awakenings throughout the night and in rapid eye movement density.

### Standard HRV Analyses.

Standard HRV measures are presented in Table 3.

Depressed patients showed significantly decreased values for the following: (1) the mean of the heartbeat interval (AVNN); (2) the mean of the s.d.'s of NN intervals in all non-overlapping 5-min segments (SDNNINDX); (3) pNN10; (4) spectral power in the VLF and LF ranges. However, multiple other commonly used measures, including the s.d. of the NN intervals (SDNN) and high frequency power (HF), were not statistically different.

### Complexity Analysis of HR Dynamics.

SampEn of the RR interval time series derived from full-night ECG recordings was higher for the healthy than for the depressed subjects across all measured time scales as shown in FIG. 1. The CI for short time scales, defined as the area under the MSE curve from scales 1 to 8 (corresponding to frequencies between 0.12 and 0.5 Hz), was significantly (P<0.04) higher for healthy (median: 17.6; range: 13.8-19.4) than depressed (median: 15.4; range: 6.9-19.8) subjects. In a regression model adjusted for age, the CI for depressed subjects was, on average, 1.8 (95% confidence interval 0.3 to 3.3, P=0.02) lower than for healthy subjects.

Although the difference between the two groups is most apparent across the shorter time scales, the healthy group still trended (P=0.09 unadjusted, P=0.06 adjusted for age) to higher dynamical HR complexity when longer time scales (1 to 20) were included in the analysis as shown in FIG. 1. To ascertain that these findings were not due to differences in the number of arousals and disrupted sleep architecture in MD, the HR complexity during different sleep states was computed. This sub-analysis was restricted to continuous ECG segments 15 min or longer because of the minimum data length requirements for multiscale signal analysis outlined above. In an effort to maximize the number of subjects included in this sub-analysis, 1 and 2, and 3 and 4 were combined.

Only 8 out of 25 (<30%) depression patients and 10 out of 20 healthy subjects had at least one 15-min or longer rapid eye movement segment. Similarly, only 10 depressed and 9 healthy subjects (<50%) had at least one 15-min or longer segment of combined sleep stages 3 and 4, precluding the use of complexity analysis for these stages. Time series of sufficient length for multiscale analysis were available in 20 healthy and 23 depressed subjects during combined sleep stages phases 1 and 2. For these non-rapid eye movement sleep periods, HR complexity was significantly higher for the control (median: 17.0; range: 7.7-30.3) than for the depression (median: 14.6; range: 9.1-28.0) group (difference of 1.43, 95% confidence interval 0.38-2.48, P =0.007, based on the generalized estimating equation model). Among depressed subjects, the CI was inversely correlated with the Hamilton rating scale for depression score (r=−0.40, P=0.05).

### Discussion

In accordance with some embodiments of the invention, the resulting complexity information can be used to detect disease as well as to assess efficacy of treatment. In each of these embodiments, one or more complexity indices can be compared to a baseline complexity as part of an assessment. In some embodiments, the subject's dynamical complexity can be compared to baseline for a comparable healthy subject, such as one or a range determined by assessing a sample population. In some embodiments, the subject's dynamical complexity can be compared to a baseline that is simply the subject's dynamical complexity determined at an earlier point in time, for example, when the subject's dynamical complexity was used to indicate a disease condition. Thus, initially, the subject's dynamical complexity can be used to indicate disease (as compared to a healthy population baseline) and then later, the subject's earlier dynamical complexity can be used as a baseline to assess response to treatment.

In accordance with the invention, acute MDEs in young to middle-aged men are associated with a decrease in cardiac interbeat interval complexity during sleeping hours as shown in FIG. 1 and measuring dynamical complexity can be used as a biomarker or indicator for MDE. This is manifest in a degradation in multiscale neuroautonomic regulation of HR over relatively short time scales (<20 s). In addition, lower CIs tend to indicate more severe depression (higher Hamilton rating scale for depression score).

Two previous studies have sought to examine changes in HR complexity with depressive illness using MSE. In one⁴⁵, the authors analyzed only daytime time series derived from relatively short (30 min) continuous ECG recordings and found that MSE for only one time scale (B3 s) was significantly higher for healthy subjects than for depressed patients. Our results differ importantly because (i) we analyze night-time recordings and (ii) we show a loss of complexity across a relatively broad range of time scales, not just a single one, which is a central requisite of the complexity-loss hypothesis.

Another study⁴⁶ assessed HR complexity during both awake and ‘bedtime’ hours in a group of patients with major depressive disorder, a group with primary insomnia and a group of healthy controls. The authors reported a reduction in complexity during sleeping but not daytime hours for both patient groups. One limitation of this study is the fact that it did not include PSG recordings and, therefore, did not control for sleep stage effects. However, these results on MD syndrome are not inconsistent with the findings reported here for patients during a major depressive episode.

Boettger et al.⁴⁷ reported on two different, putative measures of complexity in MD, including the scaling exponent b, a fractal dimension. In contrast, in this study, the observed values of this exponent (Table 3) were quantitatively consistent with previous reports for young adults^{41,42} but not statistically different between depressed and non-depressed groups.

Our findings are also of potential interest because they are aligned with the emerging concepts^{16,17,25} that (1) the dynamics of healthy systems, which show the highest adaptability, functionality and reserve, are the most complex, and (2) a variety of pathologic states,²⁸⁻³⁰ as well as advanced aging,^{48,49} are associated with loss of dynamical complexity (information-content). Our findings place MDE in this growing class of pathologic states characterized by a loss of complexity. The possible universality of complexity loss with pathology and aging may limit its diagnostic specificity. However, to the extent that complexity is a dynamical ‘assay’ of integrative (global) dynamics, measures such as MSE that probe multiscale features may provide novel ways to monitor individual patients over time and assess their response to therapeutic interventions intended to enhance functionality and plasticity.

HR complexity does not appear to be directly related to traditional HRV as assessed by measures of variance, by spectral power in selected bands, or by spectral scaling exponents. Instead, MSE appears to probe properties of the system that relate to the temporal structure of the signals it generates and not simply to the amplitude of their fluctuations. In this study, measures most directly related to the s.d. of HR time series from healthy subjects were actually comparable to those of depressed subjects (Table 3). In accordance with the invention, an important difference between the cardiac interbeat interval time series from healthy and depressed patients appears to reside in their temporal correlations, that is, the organization across, not just within, a given time scale.

In a previous study¹⁷ of HR dynamics that included healthy young and elderly subjects, as well as patients with heart disease, the authors found that a monotonic decrease in HR entropy with time scale during night time was characteristic of both healthy young and healthy elderly subjects, but not of patients with heart disease. What distinguished the former two groups was the fact that the entropy values were significantly higher for the young than for the elderly. Of further interest is the finding in the present study that the MSE profiles for the healthy and depression groups (FIG. 1) showed a monotonic decrease of entropy with time scale, qualitatively similar to the previously noted age-related changes in the MSE curves. These results, in conjunction with the fact that the MSE values were lower for the depression than the healthy group, support the putative link between the dynamics of aging and depression⁵⁰⁻⁵².

These results are also in accord with evidence that severe mood disorders are associated with excess vulnerability to cardiovascular disease, and possibly some cancers, through accelerated organismal aging.⁵² How complexity loss, interpretable as a marker of degraded system adaptability, has a role in these apparently diverse syndromes (aging, depression, cardiovascular disease) remains to be determined.

Further, measures of complexity can be useful in complementing current time and frequency domain metrics of HR dynamics. In accordance with the invention, the mean HR (inversely related to the mean interbeat interval) showed a very small but significant increase (equivalent to 6 beats per min) in the depression versus non-depression groups (Table 3). However, of the HRV measures designed to assess cardiac vagal tone modulation, only one, pNN10, was significantly reduced in the depression group. Other measures of cardiac vagal modulation (pNN25 and 50, rMSSD, HF power and LF/HF ratio) were not significantly different. In contrast, VLF and LF power, and SDNNINDX, which were significantly lower in the depression group, do not have a well-established physiologic interpretation. Overall, these HRV results are compatible with decreased vagal and increased sympathetic modulation, as previously described in depression.

Testing the diagnostic power of the complexity method and/or of any particular combination of the complexity and traditional HRV methods was outside the scope of our work. This task will require a much larger database than the one probed here.

This study has a number of limitations. First, depression is a heterogeneous syndrome, and multiple sources of variance may exist within patients (for example, severity, typicality, seasonality and number of previous episodes). Second, we excluded patients with more serious forms of psychiatric comorbidity. Third, the sample size and the methods did not allow for analysis by depressive subtypes. Advantages of this study are the restriction to unmedicated subjects with MD, not just anxiety disorder, and the exclusion of those with confounding effects due to comorbidities. Further, we examined HR dynamics not only during the entire sleeping period but also during comparable sleep phases, minimizing activity and sleep stage effects. Future studies are needed to confirm these results in larger populations, including both men and women, and, importantly, to test whether remission is associated with restoration of more complex cardiac interbeat interval dynamics.

In addition to the measuring HRV, other physiological signals, such as voice/speech and EEG signals can be used. In these embodiments, changes or features in these signals can be used to generate time series data. For speech signals, dynamical fluctuations, such as in local energy can be used to generate the time series data. For EEG signals, the features and/or energy of the signal can be used to generate the time series data. In some embodiments, the EEG signal, itself can be used, in other embodiments, time series data can be derived from high or low pass filtering of the EEG signal and/or using the sleep state classification (e.g., wake, REM, 1, 2, 3, 4) to determine the time series data.

When more that one physiological signal is available, the dynamical complexity of each signal can be quantified individually. In some embodiments, these individual measures can be analyzed using multivariate analysis techniques to assess differences between groups and provide a multi-signal indication of health or disease or recovery. Alternatively, cluster analysis can be used to determine healthy and unhealthy spaces and health (or recovery) or lack thereof can be determined by evaluating where each of the complexity values for each signal fall on the cluster graph.

In accordance with the invention, dynamical fluctuations of certain voice features, such as local energy, are less complex during depressive than normal phases. By applying a similar complexity analysis to the one outlined above, a complexity index for these signals can be determined and compared to baseline values for use in identifying mental and neurologic disease and changes in disease status or severity.

In accordance with one embodiment of the invention, “free-form” (unscripted) speech can be analyzed. In one embodiment of the invention, free-form speech from 35 subjects (consisting of 3-minute sessions; 4 sessions over a 6-week period) was compiled in a database⁵³ and analyzed using the multiscale entropy (MSE) algorithm¹⁵ to quantify the dynamical complexity of voice patterns. It should be noted that the length and number of the sessions can be greater or smaller and selected according to the desired length of the time series and the number of time scales needed.

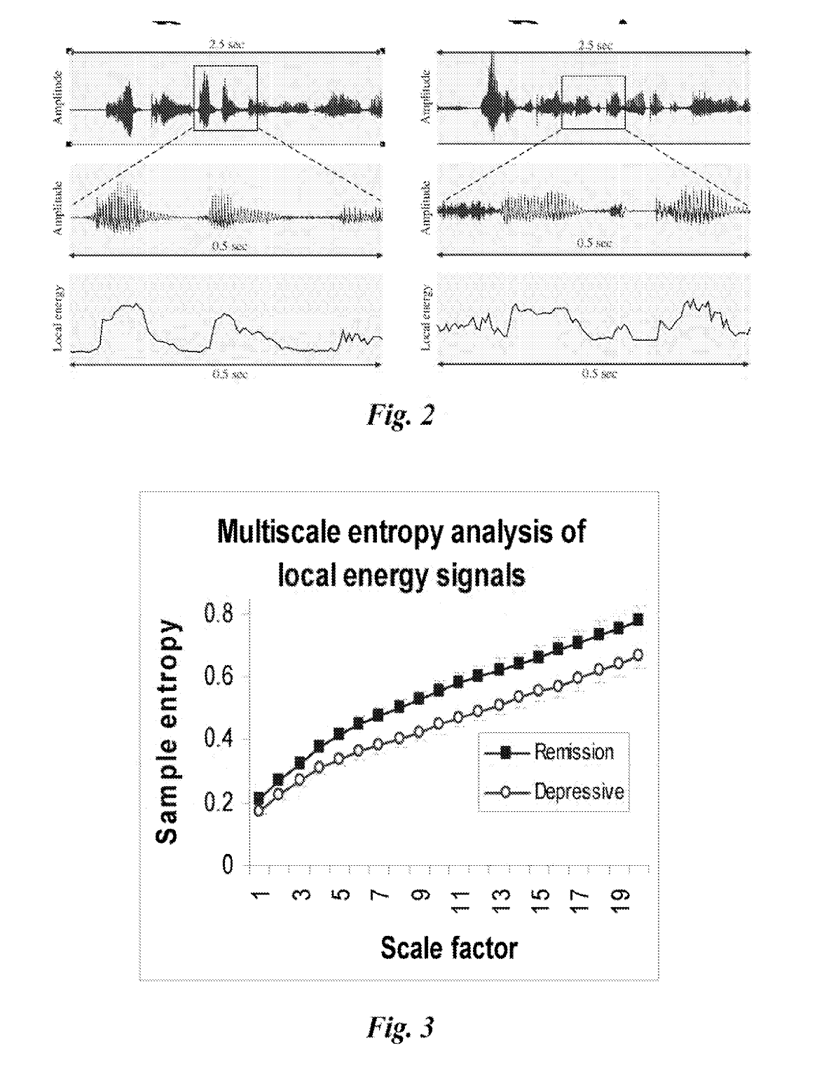

In accordance with one embodiment of the invention, the MSE computational tool quantifies the complexity of a process. It is based on the quantification of the information content of an output signal over a range of time scales. In accordance with one embodiment of the invention, a window of the voice signal corresponding to a predefined length of time can be processed to determine the local energy of the signal over the time window. For example, as shown in FIG. 2, a 0.5 second time window can be processed to determine the local energy by determining the mean square root of the amplitude over non-overlapping windows. Using these data points, a MSE analysis as described above with reference to U.S. Pat. Nos. 7,601,124 and 7,882,167 as well as references 15 and 17 identified below. Similarly, the parameter r can be determined as a function of one or more noise sources, such as the sampling frequency of the voice signal.

FIG. 2 shows the voice signal and local energy (mean square root calculated over non-overlapping windows) time series from a representative subject during the depressive and normal (remission) phases.

FIG. 3 shows the multiscale complexity analysis of local energy time series. For a broad range of time scales (1-20), information content of voice dynamics, as measured by sample entropy, is higher (p<0.005) for subjects during remission versus depressive state. (Symbols and error bars represent group mean and standard error values, respectively).

In accordance with the invention, the unscripted voice fluctuations in major depression appear less complex than those during remission and complexity of voice can be used as a biomarker of major depression, including use in diagnosis, follow-up, and therapeutic assessment.

Further, other multiscale nonlinear/complexity measures can also be used as adjuncts to biomarker development in depression and related syndromes.

Other embodiments are within the scope and spirit of the invention. For example, due to the nature of software, functions described above can be implemented using software, hardware, firmware, hardwiring, or combinations of any of these. Features implementing functions may also be physically located at various positions, including being distributed such that portions of functions are implemented at different physical locations.

Further, while the description above refers to the invention, the description may include more than one invention.

Although some of various drawings illustrate a number of logical stages in a particular order, stages which are not order dependent can be reordered and other stages can be combined or broken out. Alternative orderings and groupings, whether described above or not, can be appropriate or obvious to those of ordinary skill in the art of computer science. Moreover, it should be recognized that the stages could be implemented in hardware, firmware, software or any combination thereof.

The foregoing description, for purpose of explanation, has been described with reference to specific embodiments. However, the illustrative discussions above are not intended to be exhaustive or to be limiting to the precise forms disclosed. Many modifications and variations are possible in view of the above teachings. The embodiments were chosen and described in order to best explain the principles of the aspects and its practical applications, to thereby enable others skilled in the art to best utilize the aspects and various embodiments with various modifications as are suited to the particular use contemplated.

The following references are incorporated herein by reference in their entirety
1. Kessler R C, Berglund P, Demler O, Jin R, Koretz D, Merikangas K R et al. The epidemiology of major depressive disorder: results from the National Comorbidity Survey Replication (NCS-R). JAMA 2003; 289: 3095-3105.
2. Hasin D S, Goodwin R D, Stinson F S, Grant B F. Epidemiology of major depressive disorder: results from the National Epidemiologic Survey on Alcoholism and Related Conditions. Arch Gen Psychiatry 2005; 62: 1097-1106.
3. Murray C J, Lopez A D. Alternative projections of mortality and disability by cause 1990-2020: Global Burden of Disease Study. Lancet 1997; 349: 1498-1504.
4. Musselman D L, Evans D L, Nemeroff C B. The relationship of depression to cardiovascular disease. Arch Gen Psychiatry 1998; 55: 580-592.
5. Belmaker R H, Agam G. Major depressive disorder. N Engl J Med 2008; 358: 55-68.
6. Penninx B W J H, Beekman A T F, Honig A, Deeg D J H, Schoevers R A, van Eijk J T M et al. Depression and cardiac mortality: results from a community-based longitudinal study. Arch Gen Psychiatry 2001; 58: 221-227.
7. Lesperance F, Frasure-Smith N, Talajic M, Bourassa M G. Five-year risk of cardiac mortality in relation to initial severity and one-year changes in depression symptoms after myocardial infarction. Circulation 2002; 105: 1049-1053.
8. Carney R M, Freedland K E, Miller G E, Jaffe A S. Depression as a risk factor for cardiac mortality and morbidity: a review of potential mechanisms. J Psychosom Res 2002; 53:897-902.
9. Parissis J T, Fountoulaki K, Filippatos G, Adamopoulos S, Paraskevaidis I, Kremastinos D. Depression in coronary artery disease: novel pathophysiologic mechanisms and therapeutic implications. Int J Cardiol 2007; 116: 153-160.
10. American Psychiatric Association. Diagnostic and Statistical Manual of Mental Disorders, 4th edn, DSM-IV-TR. (text revision). American Psychiatric Association: Washington D.C., 2000.
11. Steiger A, Kimura M. Wake and sleep EEG provide biomarkers in depression. J Psychiatr Res 2010; 44: 242-252.
12. Miller A H, Maletic V, Raison C L. Inflammation and its discontents: the role of cytokines in the pathophysiology of major depression. Biol Psychiatry 2009; 65: 732-741.
13. Samuels A M. The brain-heart connection. Circulation 2007; 116: 77-84.
14. Valentini M, Parati G. Variables influencing heart rate. Prog Cardiovasc Dis 2009; 52: 11-19.
15. Costa M, Goldberger A L, Peng C-K. Multiscale entropy analysis of complex physiologic time series. Phys Rev Lett 2002; 89: 068102-1-068102-4.
16. Chialvo D R. Physiology: unhealthy surprises. Nature 2002; 419: 263.
17. Costa M, Goldberger A L, Peng C-K. Multiscale entropy analysis of biological signals. Phys Rev E Stat Nonlin Soft Matter Phys 2005; 71: 021906-1-18.
18. Yeragani V K. Major depression and long-term heart period variability. Depress Anxiety 2000; 12: 51-52.
19. Yeragani V K, Rao K A, Smitha M R, Pohl R B, Balon R, Srinivasan K. Diminished chaos of heart rate time series in patients with major depression. Biol Psychiatry 2002; 51: 733-744.
20. Glassman A H, Bigger J T, Gaffney M, Van Zyl L T. Heart rate variability in acute coronary syndrome patients with major depression: influence of sertraline and mood improvement. Arch Gen Psychiatry 2007; 64: 1025-1031.
21. Licht C M M, de Geus E J C, Zitman F G, Hoogendijk W J G, van Dyck R, Penninx B W J H. Association between major depressive disorder and heart rate variability in the Netherlands Study of Depression and Anxiety (NESDA). Arch Gen Psychiatry 2008; 65: 1358-1367.
22. Servant D, Logier R, Mouster Y, Goudemand M. Heart rate variability. Applications in psychiatry. Encephale 2009; 35: 423-428.
23. Kemp A H, Quintana D S, Gray M A, Felmingham K L, Brown K, Gatt J M. Impact of depression and antidepressant treatment on heart rate variability: a review and metaanalysis. Biol Psychiatry 2010; 67: 1067-1074.
24. Grassberger P. Information and Complexity Measures in Dynamical Systems. In: Atmanspacher H, Scheingraber H (eds). Information Dynamics. Plenum Press: New York, 1991 pp 15-33.
25. Goldberger A L, Giles F. Filley lecture. Complex systems. Proc Am Thorac Soc 2006; 3: 467-472.
26. Goldberger A L, Amaral L A, Hausdorff J M, Ivanov PCh, Peng C-K, Stanley H E. Fractal dynamics in physiology: alterations with disease and aging. Proc Natl Acad Sci USA 2002; 19 (Suppl 1): 2466-2472.
27. Goldberger A, Peng C-K, Lipsitz L A. What is physiologic complexity and how does it change with aging and disease? Neurobiol Aging 2002; 23: 23-26.
28. Norris P R, Anderson S M, Jenkins J M, Williams A E, Morris Jr J A. Heart rate multiscale entropy at three hours predicts hospital mortality in 3,154 trauma patients. Shock 2008; 30: 17-22.
29. Riordan Jr W P, Norris P R, Jenkins J M, Morris Jr J A. Early loss of heart rate complexity predicts mortality regardless of mechanism, anatomic location, or severity of injury in 2178 trauma patients. J Surg Res 2009; 156: 283-289.
30. Ferrario M, Signorini M G, Magenes G. Complexity analysis of the fetal heart rate variability: early identification of severe intrauterine growth-restricted fetuses. Med Biol Eng Comput 2009; 47: 911-919.
31. Richman J S, Moorman J R. Physiological time series analysis using approximate entropy and sample entropy. Am J Physiol 2000; 278: H2039-H2049.
32. Pincus S M. Assessing serial irregularity and its implications for health. Ann NY Acad Sci 2001; 954: 245-267.
33. Task Force of the European Society of Cardiology the North American Society of Pacing Electrophysiology. Heart rate variability: standards of measurement, physiological interpretation, and clinical use. Circulation 1996; 93: 1043-1065.
34. Hamilton M. A rating scale for depression. J Neurol Neurosurg 1960; 23: 56-62.
35. Buysse D J, Reynolds C F, Monk T H, Berman S R, Kupfer D J. The Pittsburgh sleep quality index: a new instrument for psychiatric practice and research. Psychiatry Res 1989; 28: 193-213.
36. Leistedt SJ-J, Coumans N, Dumont M, Lanquart J-P, Stam C J, Linkowski P. Altered sleep brain functional connectivity in acutely depressed patients. Hum Brain Mapp 2009; 30:2207-2219.
37. Agnew Jr H W, Webb W B, Williams R L. The first night effect: an EEG study of sleep. Psychophysiology 1966; 2: 263-266.
38. Rechtschaffen A, Kales A (eds) A manual of standardized terminology techniques and scoring system for sleep stages of human subjects. UCLA Brain Information Service/Brain Research Institute, University of California: Los Angeles, Calif., 1968.
39. Devlin P, Moody G, Mark R. Desing and evaluation of amodular real-time QRS detector. Proc 35th Ann Conf on Engineering in Medicine and Biology, Bethesda, USA; 1982; 40.
40. Mietus J E, Peng C K, Henry I, Goldsmith R L, Goldberger A L. The pNNx-files: re-examining a widely-used heart rate variability measure. Heart 2002; 88: 378-380.
41. Saul J P, Albrecht P, Berger R D, Cohen R J. Analysis of long term heart rate variability: meth-ods, 1/f scaling and implications. Comput Cardiol 1988; 14: 419-422.
42. Bigger J T, Steinman R C, RoInitzky L M, Fleiss J L, Albrecht P, Cohen R J. Power law behavior of RR-interval variability in healthy middle-aged persons, patients with recent acute myocardial infarction, and patients with heart transplants. Circulation 1996; 93: 2142-2151.
43. Kerkhofs M, Linkowski P, Lucas F, Mendlewicz J. Twenty-four-hours patterns of sleep in depression. Sleep 1991; 14: 501-506.
44. Kupfer D J. Sleep research in depressive illness: clinical implications—a tasting menu. Biol Psychiatry 1995; 38: 391-403.
45. Schulz S, Koschke M, Bar K-J, Voss A. The altered complexity of cardiovascular regulation in depressed patients. Physiol Meas 2010; 31: 303-321.
46. Yang A C, Tsai S J, Yang C-H, Kuo C-H, Chen T-J, Hong C-J. Reduced physiologic complexity is associated with poor sleep in patients with major depression and primary insomnia. J Affect Disord, advanced online publication 2010 e-pub ahead of print.
47. Boettger S, Hoyer D, Falkenhahn K, Kaatz M, Yeragani V K, Bar K-J. Nonlinear broad band dynamics are less complex in major depression. Bipolar Disord 2008; 10: 276-284.
48. Jokinen V, Syvae~nne M, Mae~ kikallio T H, Airaksinen K E J, Huikuri H V. Temporal agerelated changes in spectral, fractal and complexity characteristics of heart rate variability. Clin Physiol 2001; 21: 273-281.
49. Lipsitz L A. Aging as a Process of Complexity Loss. In: Deisboeck T S, Kresh J Y (eds). Topics in Biomedical Engineering International Book Series: Complex Systems Science in Biomedicine. Springer, 2006 pp 641-654.
50. Starr J M, McGurn B, Harris S E, Whalley L J, Deary I J, Shiels P G. Association between telomere length and heart disease in a narrow age cohort of older people. Exp Gerontol 2007; 42: 571-573.
51. Epel E S, Blackburn E H, Lin J, Dhabhar F S, Adler N E, Morrow J D et al. Accelerated telomere shortening in response to life stress. Proc Natl Acad Sci USA 2004; 101: 17312-17315.
52. Simon N M, Smoller J W, McNamara K L, Maser R S, Zalta A K, Pollack M H et al. Telomere shortening and mood disorders: preliminary support for a chronic stress model of accelerating aging. Biol Psychiatry 2006; 60: 432-435.
53. Mundt J C et al. Voice acoustic measures of depression severity and treatment response collected via interactive voice response (IVR) technology. Journal of Neurolinguistics 20 (2007) 50-64.

1. A method for detecting a mental disease comprising:
measuring at least one physiological signal from a patient;
determining a signal complexity index as a function of at least one of the physiological signals; and
comparing the signal complexity index to at least one of a baseline complexity index of a healthy subject, a baseline complexity index of the patient prior to treatment or a baseline complexity index of the patient at a past time to determine if the signal complexity index varies from the baseline complexity index by a threshold amount.

2-48. (canceled)

49. The method according to claim 1 wherein determining the signal complexity index includes determining an entropy value over multiple time scales.

50. The method according to claim 49 wherein determining the signal complexity includes determining a tolerance parameter r and the tolerance parameter r is determined as a function of a sampling frequency of at least one of the physiological signals.

51. The method according to claim 1 wherein the physiological signals are selected from the group including heart rate signals, brain wave signals and voice signals.

52. The method according to claim 1, the method further comprising:
developing a treatment plan for the mental disease based on the variation between the signal complexity index and the baseline complexity index.

53. The method according to claim 1 wherein the signal complexity index is a short time scale signal complexity index, wherein the short time scale signal complexity index corresponds with an area under a multiscale entropy curve from scales 1 to 8 corresponding to frequencies between 0.12 and 0.5 Hz, and wherein the short time scale signal complexity index is compared to an average short time scale complexity index for the healthy subject.

54. The method according to claim 53, wherein the mental disease is indicated when the short time scale signal complexity index of the patient is statistically significantly lower than the average short time scale complexity index for the healthy subject.

55. The method according to claim 54, wherein the mental disease is indicated when the short time scale signal complexity index of the patient is about 1.8 lower than the average short time scale complexity index for the healthy subject.

56. The method according to claim 1, wherein a lower signal complexity index indicates a more severe mental disease as compared to a higher signal complexity index.

57. The method of claim 1, wherein the mental disease is indicated by a statistically significantly lower value of at least one of a mean of a heartbeat interval, a mean of a standard deviation of normal sinus to normal sinus (NN) intervals in all non-overlapping 5-minute segments, a percentage of adjacent NN intervals whose difference is higher than 10 ms and a spectral power in very low frequency and low frequency ranges.

58. The method of claim 1, wherein the mental disease is major depression.

59. The method according to claim 1, the method further comprising:
modifying an existing treatment plan for the mental disease based on the variation between the signal complexity index and the baseline complexity index.

60. A computer implemented method for detecting a mental disease, comprising:
on a device having one or more processors and a memory storing one or more programs for execution by the one or more processors, the one or more programs including instructions for:
measuring at least one physiological signal from a patient;
determining a signal complexity index as a function of at least one of the physiological signals; and
comparing the signal complexity index to at least one of a baseline complexity index of a healthy subject, a baseline complexity index of the patient prior to treatment or a baseline complexity index of the patient at a past time to determine if the signal complexity index varies from the baseline complexity index by a threshold amount.

61. The computer implemented method according to claim 60 wherein determining the signal complexity index includes determining an entropy value over multiple time scales.

62. The computer implemented method according to claim 61 wherein determining the signal complexity includes determining a tolerance parameter r and the tolerance parameter r is determined as a function of a sampling frequency of at least one of the physiological signals.

63. The computer implemented method according to claim 60 wherein the physiological signals are selected from the group including heart rate signals, brain wave signals and voice signals.

64. The computer implemented method according to claim 60, the computer implemented method further comprising:
developing a treatment plan for the mental disease based on the variation between the signal complexity index and the baseline complexity index.

65. The computer implemented method according to claim 60 wherein the signal complexity index is a short time scale signal complexity index, wherein the short time scale signal complexity index corresponds with an area under a multiscale entropy curve from scales 1 to 8 corresponding to frequencies between 0.12 and 0.5 Hz, and wherein the short time scale signal complexity index is compared to an average short time scale complexity index for the healthy subject.

66. The computer implemented method according to claim 65, wherein the mental disease is indicated when the short time scale signal complexity index of the patient is statistically significantly lower than the average short time scale complexity index for the healthy subject.

67. The computer implemented method according to claim 66, wherein the mental disease is indicated when the short time scale signal complexity index of the patient is about 1.8 lower than the average short time scale complexity index for the healthy subject.

68. The computer implemented method according to claim 60, wherein a lower signal complexity index indicates a more severe mental disease as compared to a higher signal complexity index.

69. The computer implemented method of claim 60, wherein the mental disease is indicated by a statistically significantly lower value of at least one of a mean of a heartbeat interval, a mean of a standard deviation of normal sinus to normal sinus (NN) intervals in all non-overlapping 5-minute segments, a percentage of adjacent NN intervals whose difference is higher than 10 ms and a spectral power in very low frequency and low frequency ranges.

70. The computer implemented method of claim 60, wherein the mental disease is major depression.

71. The computer implemented method according to claim 60, the computer implemented method further comprising:
modifying an existing treatment plan for the mental disease based on the variation between the signal complexity index and the baseline complexity index.

72. A computer system for detecting a mental disease, comprising:
one or more processors; and
memory to store:
one or more programs, the one or more programs comprising:
instructions for:
measuring at least one physiological signal from a patient;
determining a signal complexity index as a function of at least one of the physiological signals; and
comparing the signal complexity index to at least one of a baseline complexity index of a healthy subject, a baseline complexity index of the patient prior to treatment or a baseline complexity index of the patient at a past time to determine if the signal complexity index varies from the baseline complexity index by a threshold amount.

73. The computer system according to claim 72 wherein determining the signal complexity index includes determining an entropy value over multiple time scales.

74. The computer system according to claim 73 wherein determining the signal complexity includes determining a tolerance parameter r and the tolerance parameter r is determined as a function of a sampling frequency of at least one of the physiological signals.

75. The computer system according to claim 72 wherein the physiological signals are selected from the group including heart rate signals, brain wave signals and voice signals.

76. The computer system according to claim 72, wherein the one or more programs further comprise instructions for:
developing a treatment plan for the mental disease based on the variation between the signal complexity index and the baseline complexity index.

77. The computer system according to claim 72 wherein the signal complexity index is a short time scale signal complexity index, wherein the short time scale signal complexity index corresponds with an area under a multiscale entropy curve from scales 1 to 8 corresponding to frequencies between 0.12 and 0.5 Hz, and wherein the short time scale signal complexity index is compared to an average short time scale complexity index for the healthy subject.

78. The computer system according to claim 77, wherein the mental disease is indicated when the short time scale signal complexity index of the patient is statistically significantly lower than the average short time scale complexity index for the healthy subject.

79. The computer system according to claim 78, wherein the mental disease is indicated when the short time scale signal complexity index of the patient is about 1.8 lower than the average short time scale complexity index for the healthy subject.

80. The computer system according to claim 72, wherein a lower signal complexity index indicates a more severe mental disease as compared to a higher signal complexity index.

81. The computer system of claim 72, wherein the mental disease is indicated by a statistically significantly lower value of at least one of a mean of a heartbeat interval, a mean of a standard deviation of normal sinus to normal sinus (NN) intervals in all non-overlapping 5-minute segments, a percentage of adjacent NN intervals whose difference is higher than 10 ms and a spectral power in very low frequency and low frequency ranges.

82. The computer system of claim 72, wherein the mental disease is major depression.

83. The computer system according to claim 72, wherein the one or more programs further comprise instructions for:
modifying an existing treatment plan for the mental disease based on the variation between the signal complexity index and the baseline complexity index.

84. A non-transitory computer-readable storage medium storing one or more programs configured to be executed by one or more processing units at a computer comprising:
instructions for detecting a mental disease:
measuring at least one physiological signal from a patient;
determining a signal complexity index as a function of at least one of the physiological signals; and
comparing the signal complexity index to at least one of a baseline complexity index of a healthy subject, a baseline complexity index of the patient prior to treatment or a baseline complexity index of the patient at a past time to determine if the signal complexity index varies from the baseline complexity index by a threshold amount.

85. The computer system according to claim 84 wherein determining the signal complexity index includes determining an entropy value over multiple time scales.

86. The non-transitory computer-readable storage medium according to claim 85 wherein determining the signal complexity includes determining a tolerance parameter r and the tolerance parameter r is determined as a function of a sampling frequency of at least one of the physiological signals.

87. The non-transitory computer-readable storage medium according to claim 84 wherein the physiological signals are selected from the group including heart rate signals, brain wave signals and voice signals.

88. The non-transitory computer-readable storage medium according to claim 84, wherein the one or more programs further comprise instructions for:
developing a treatment plan for the mental disease based on the variation between the signal complexity index and the baseline complexity index.

89. The non-transitory computer-readable storage medium according to claim 84 wherein the signal complexity index is a short time scale signal complexity index, wherein the short time scale signal complexity index corresponds with an area under a multiscale entropy curve from scales 1 to 8 corresponding to frequencies between 0.12 and 0.5 Hz, and wherein the short time scale signal complexity index is compared to an average short time scale complexity index for the healthy subject.

90. The non-transitory computer-readable storage medium according to claim 89, wherein the mental disease is indicated when the short time scale signal complexity index of the patient is statistically significantly lower than the average short time scale complexity index for the healthy subject.

91. The non-transitory computer-readable storage medium according to claim 90, wherein the mental disease is indicated when the short time scale signal complexity index of the patient is about 1.8 lower than the average short time scale complexity index for the healthy subject.

92. The non-transitory computer-readable storage medium according to claim 84, wherein a lower signal complexity index indicates a more severe mental disease as compared to a higher signal complexity index.

93. The non-transitory computer-readable storage medium of claim 84, wherein the mental disease is indicated by a statistically significantly lower value of at least one of a mean of a heartbeat interval, a mean of a standard deviation of normal sinus to normal sinus (NN) intervals in all non-overlapping 5-minute segments, a percentage of adjacent NN intervals whose difference is higher than 10 ms and a spectral power in very low frequency and low frequency ranges.

94. The non-transitory computer-readable storage medium of claim 84, wherein the mental disease is major depression.

95. The non-transitory computer-readable storage medium according to claim 84, wherein the one or more programs further comprise instructions for:
modifying an existing treatment plan for the mental disease based on the variation between the signal complexity index and the baseline complexity index.
